(19) United States
(12) Patent Application Publication
(10) Pub. No.: US 2004/0039161 A1
(43) Pub. Date: Feb. 26, 2004

(54) Use of trichloroacetimidate linker for peptide synthesis

(76) Inventors: John Philip Mayer, Indianapolis, IN (US); Liang Zeng Yan, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/225,493

(22) Filed: Aug. 22, 2002

(51) Int. Cl. C07K 1/02

(52) U.S. Cl. 530/333

(57) ABSTRACT

The present invention provides a solid phase peptide synthetic method for the preparation of C-terminal alchohols, wherein the improvement consists of using trichloroacetimidate as the linker.

### BACKGROUND OF THE INVENTION

The conventional method of preparing peptides with a C-terminal alcohol involves either solution-phase synthesis, which is not practical for peptides longer than 10 residues, or solid-phase peptide synthesis. The successful execution of a solid-phase procedure requires the covalent attachment of the starting material to a solid support, usually polystyrene or polyethylene glycol, through an appropriate linker or “handle”. The classic C-terminal anchoring strategy for peptide synthesis based on benzyl and benzhydrylamine linkers for Boc/Bzl (tert-butoxycarbonyl/benzyl) chemistry as well as the more labile alkoxybenzyl or 2,4-dimethoxybenzhydrylamine versions used in Fmoc/tBu (fluorenylmethoxycarbonyl/tert-butyl) protocols are generally restricted in their application to the synthesis of peptides with either free carboxyl or carboxyl amide termini. (Merrifield, R B, J. Am. Chem. Soc. 1963, 86, 304.; Mitchell, et.al., J. Am. Chem. Soc. 1976, 98, 7357-7362.; Matsueda, et. al., Peptides, 1981, 2, 45-50.; Wang, S S, J. Am. Chem. Soc. 1972, 95, 1328-1333.; Rink, H, Tet. Let., 1987, 28, 3787-3790.)

Although C-terminal anchoring permits modifications to the N-terminus of a peptide, it is not very useful for modifications to the C-terminus itself, as is required for C-terminal alcohols. Modifications to the C-terminus itself tend to be more challenging and may require use of specialized linkers, prior cleavage from support or other indirect methods. Examples of such modifications are: a C-terminal alcohol shared by the antibacterial peptide alamethicin (Mueller, et al., Nature, 1968, 217, 713-719.), a number of cholecystokinin antagonists (Horwell, et al., J. Med. Chem., 1991, 34, 404-414.), growth hormone secretagogues (Ankerson, et al., J. Med. Chem., 1998, 41, 3169-3704.), and the growth hormone inhibitor, octreotide (Bauer, et al, J. Life Sci., 1982, 31, 1133-1140.).

The use of alternative linkers is the most versatile approach to addressing this synthetic problem. Trityl chloride is one such specialized linker. However, this linker is relatively expensive at $10-20 US/gram, and the loading of the amino acid alcohol is extremely sluggish and inefficient (15% capacity after 1-2 days). There is a fundamental need for a more cost effective and efficient linker to be discovered. Surprisingly and unexpectedly, we have now found that the use of trichloroacetimidate modified Wang resin to carry out solid-phase synthesis of peptides with a C-terminus alcohol moiety is inexpensive, about $2-5 US/gram, loads extremely rapidly and efficiently, 70% capacity in 1 hour, and can be recycled. Although the trichloroacetimidate derivative of Wang resins has previously been used as polymer-bound benzylating reagents for a variety of organic small molecules, (Hanessian, S., Xie, F. Tetrahedron Letters 1998, 39, 733-736.), its use in peptide chemistry was heretofore unknown.

### BRIEF SUMMARY OF THE INVENTION

The present invention provides a solid phase peptide synthetic method for the preparation of C-terminal alcohols, wherein the improvement is the use of trichloroacetimidate activated linker.

The present invention relates to a process for preparing a C-terminal amino alcohol containing peptide comprising: a. exposing the trichloroacetimidate modified Wang resin to an appropriate Fmoc protected amino alcohol to form a solid phase support; b. reacting the support with an amino acid residue to form an attached peptide; c. optionally repeating step b; and d. cleaving the attached peptide to form said C-terminal amino alcohol containing peptide. Preferably, the C-terminal amino alcohol containing peptide is octreotide.

Furthermore, the present invention relates to a process for preparing a solid-phase support for the synthesis of a C-terminal amino alcohol containing peptide comprising: exposing trichloroacetimidate modified Wang resin to an appropriate Fmoc protected amino alcohol to form said solid-phase support.

Furthermore, the present invention relates to a process for preparing octreotide comprising: a. exposing the trichloroacetimidate modified Wang resin to Fmoc-Thr(tBut)-ol to form a solid phase support; b. reacting the support with Thr(OtBu) to form an attached peptide; c. optionally repeating step b sequentially with the following optionally protected amino acid residues: Cys, Thr, Lys, Trp, Phe, Cys, and Phe; and d. cleaving the attached peptide to form octreotide.

### DETAILED DESCRIPTION OF THE INVENTION

The term “C-terminal amino alcohol containing peptide” refers to a peptide in which the C-terminus is an α or β amino alcohol.

The term “trichloroacetimidate modified Wang resin” refers to a Wang resin that has been activated by trichloroacetimidate.

An “appropriate Fmoc protected amino alcohol” refers to an amino alcohol protected with Fmoc. Appropriate amino alcohols include, but are not limited to, glycinol, alaninol, D-alaninol, argininol(Pbf), leucinol, phylalaninol, d-phenylalaninol, prolinol, tryptohanol, lysinol(tBoc), and threoninol(OtBu).

The term “optionally protected amino acid residue” refers to an amino acid residue that has optionally been protected. A skilled artisan would appreciate that a protecting group may or may not be required, depending on the amino acid and the reaction conditions, and additionally, the protecting group may vary depending on the particular amino acid. Such optionally protected amino acids include, but are not limited to, Thr(OtBu), Lys(Boc), Trp(Boc), Cys(Trt), Ala, Arg, Asn, Asp, Gln, Glu, Gly, His, Ile, Leu, Met, Phe, Pro, Ser, Tyr, and Val.

The term “suitable solvent” refers to any solvent, or mixture of solvents, inert to the ongoing reaction that sufficiently solubilizes the reactants to afford a medium within which to effect the desired reaction.

The present invention provides a process for preparing a solid-phase support for the synthesis of a C-terminal amino alcohol containing peptide consisting of exposing trichloroacetimidate modified Wang resin to an appropriate Fmoc protected amino alcohol to form said solid-phase support as illustrated in Scheme 1. This reaction is generically described in Scheme 1, where in R1 is the tail of the appropriate amino acid:

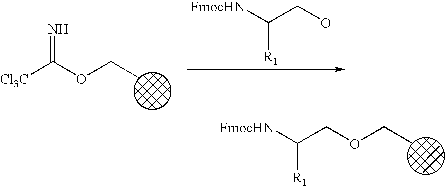

The trichloroacetimidate modified Wang resin is exposed to an Fmoc protected amino alcohol in the presence of a Lewis acid in a suitable solvent. Preferably, the resin is exposed to a three-fold excess of the Fmoc protected amino alcohol in a suitable solvent in the presence of a Lewis acid, i.e. 0.2 eq. of boron trifluoride diethyl etherate, for one hour.

Fmoc protected amino alcohols are commercially available or may be prepared according to the procedure of Rodriquez, et al. Tetrahedron Letters, 1991, 32, 923-926. Suitable solvents for the present reaction are those that provide reasonable solubility for the reactants, such as dichloromethane, diether ether, and tetrahydropyran. Preferably the solvent is dry THF (tetrahydrofuran). An excess of about 2 to about 4 equivalents of the protected amino alcohol is preferred. About a 3-fold excess of the Fmoc amino alcohol is more preferred.

Modified Wang resins may be prepared by methods well known in the art, see for example Hanessian, S., et. al. Tetrahedron Letters 1998, 39, 733-736. Generically, the trichloroacetimidate modified Wang resin is prepared by adding trichloroacetonitrile (1.5 mL) to a suspension of Wang resin (0.8 mmol) in dry CH₂Cl₂ (10 mL) and then cooling to 0° C. Add to this mixture DBU (0.1 mL) dropwise over a period of 5 minutes. Allow reaction to proceed for 40 minutes at 0° C. Collect the resin on a sintered glass filter and wash with CH₂Cl₂, DMSO, THF, and CH₂Cl₂.

The quantity of Lewis acid is not critical to the reaction. It is preferred that about 0.5 equivalent of boron trifluoride diethyl etherate is used towards trichloroacetimidate Wang resin to provide optimum loading, however, excess of the Lewis acid neither benefits nor hinders the reaction. However, the skilled artisan would appreciate that an acceptable Lewis acid must be suitable with the selected reactants in the selected reaction conditions. Preferably the Lewis acid selected for this would be boron trifluoride diethyl etherate. Aluminum trichloride anhydrous (AlCl₃) can be used as the Lewis acid catalyst using for the initial loading of amino alcohols. However, it is less efficient than boron trifluoride etherate (BF₃.Et₂O). It required one equivalent of AlCl₃ in order to achieve reasonable loading. See Tables 1 and 2.

TABLE 1
Loading using different amount of aluminum trichioride (RT, 60 min)

| AlCl₃ | Resin Wt | | | | | Loading |
|---|---|---|---|---|---|---|
| (eg) | (mg) | OD₃₀₁ₙₘ | | | | (mmole/g) |
| 0.1 | 7.5 | 0.022 | 0.021 | 0.019 | 0.019 | 0.013 |
| 0.2 | 6.9 | 0.038 | 0.038 | 0.039 | 0.038 | 0.026 |
| 0.5 | 6.7 | 0.139 | 0.139 | 0.138 | 0.138 | 0.096 |
| 1.0 | 6.4 | 0.304 | 0.304 | 0.303 | 0.304 | 0.22 |
| 2.0 | 6.2 | 0.292 | 0.290 | 0.290 | 0.288 | 0.22 |

TABLE 2
Loading using different time with AlCl₃ (RT, AlCl₃ 0.5 eq)

| Time | Resin Wt | | | | | Loading |
|---|---|---|---|---|---|---|
| (min) | (mg) | OD₃₀₁ₙₘ | | | | (mmole/g) |
| 5 | 7.2 | 0.152 | 0.152 | 0.153 | 0.153 | 0.097 |
| 10 | 7.4 | 0.122 | 0.122 | 0.121 | 0.121 | 0.076 |
| 30 | 5.4 | 0.102 | 0.103 | 0.102 | 0.103 | 0.087 |
| 60 | 6.1 | 0.114 | 0.116 | 0.116 | 0.114 | 0.087 |
| 120 | 7.1 | 0.138 | 0.140 | 0.140 | 0.140 | 0.091 |
| overnight | 6.5 | 0.147 | 0.147 | 0.147 | 0.147 | 0.10 |

Loading of the protected amino alcohols onto Wang resin is done by methods well known to the skilled artisan, see for example, Hanessian, S., Xie, F. Tetrahedron Letters 1998, 39, 733-736. This loading is completed in about 5 to about 60 minutes. The skilled artisan would appreciate that some amino acids and conditions will complete in less than an hour and some will take longer than an hour. However, the skilled artisan would appreciate that a prolonged reaction time would not improve the loading efficiency. For example, using BF₃.Et₂O, the reaction was completed almost immediately, even though the reaction could be left overnight without any deleterious effects (Table 3). Small amount of BF₃.Et₂O (0.05 eq) can provide efficient catalytic activity (Table 4).

TABLE 3
Loading using different time with BF₃ · Et₂O (RT, BF₃ · Et₂O 0.5 eq)

| | Resin Wt | | | | | Loading |
|---|---|---|---|---|---|---|
| Time | (mg) | OD_{301nm (mmole/g)} | | | | |
| 1 min | 7.1 | 0.710 | 0.711 | 0.709 | 0.712 | 0.46 |
| 5 min | 6.3 | 0.627 | 0.627 | 0.630 | 0.630 | 0.46 |
| 10 min | 6.5 | 0.610 | 0.611 | 0.610 | 0.612 | 0.43 |
| 20 min | 5.5 | 0.549 | 0.548 | 0.547 | 0.547 | 0.46 |
| 30 min | 5.9 | 0.597 | 0.597 | 0.598 | 0.598 | 0.47 |
| 1 h | 6.0 | 0.629 | 0.628 | 0.630 | 0.629 | 0.48 |
| 2 h* | 5.6 | 0.615 | 0.612 | 0.612 | 0.615 | 0.45 |
| 4 h | 5.7 | 0.624 | 0.620 | 0.624 | 0.620 | 0.45 |
| 8 h | 5.6 | 0.590 | 0.590 | 0.591 | 0.591 | 0.44 |
| 24 h | 6.2 | 0.665 | 0.665 | 0.660 | 0.660 | 0.45 |
| 30 h | 5.2 | 0.550 | 0.550 | 0.548 | 0.550 | 0.44 |

*Data at 2 h to 30 h were collected at a different time.

TABLE 4
Loading using different amount of BF₃ · Et₂O (RT, 60 min)

| BF₃ · Et₂O | Resin Wt | | | | | Loading |
|---|---|---|---|---|---|---|
| (eg) | (mg) | OD₃₀₁ₙₘ | | | | (mmole/g) |
| 0.00 | 7.3 | 0.010 | 0.010 | 0.012 | 0.012 | 0.006 |
| 0.01 | 6.9 | 0.232 | 0.232 | 0.230 | 0.231 | 0.15 |
| 0.05 | 7.0 | 0.707 | 0.708 | 0.712 | 0.712 | 0.47 |
| 0.1 | 7.8 | 0.797 | 0.798 | 0.798 | 0.798 | 0.47 |
| 0.2 | 6.6 | 0.671 | 0.673 | 0.675 | 0.675 | 0.47 |
| 0.5 | 6.6 | 0.666 | 0.666 | 0.673 | 0.672 | 0.47 |
| 1.0 | 7.2 | 0.752 | 0.751 | 0.752 | 0.752 | 0.48 |

After the support is complete, the support is reacted with a series of amino acid residues to form an attached peptide, as is shown in scheme II, by traditional direct solid-phase synthesis techniques known in the art, which is then cleaved by methods well known in the art, wherein R1 and Rn are the tails of appropriate amino acids.

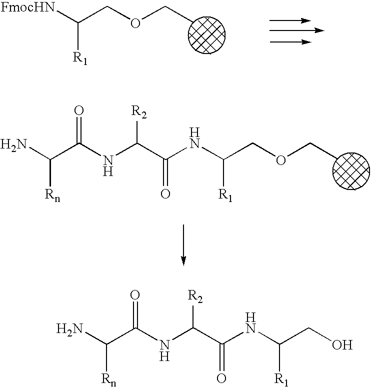

Experimental

### Preparation I

### Modified Wang Resin

Add trichloroacetonitrile (1.5 mL) to a suspension of Wang resin (0.8 mmol) in dry dichloromethane (CH₂Cl₂) (10 mL) and then cool to 0° C. Add to this mixture 1,8-diazabicyclo[5.4.0]undec-7-ene (DBU) (0.1 mL) dropwise over a period of 5 minutes. Allow reaction to proceed for 40 minutes at 0° C. Collect the resin on a sintered glass filter and wash with CH₂Cl₂, DMSO, THF, and CH₂Cl₂.

### Preparation II

### Coupling of Wang Resin with Aminoalcohol

Trichloroacetimidate Wang resin (1.3 g, 1.0 mmole, 0.77 mmole/g) was swollen in dichloromethane for 30 min. The resin was then washed with dry THF a few times. Fmoc-Thr(tBut)-ol (1.15 g, 3.0 mmoles) was dissolved in 20 mL of dry THF and transferred to the THF washed resin. The resin with the amino alcohol solution was briefly mixed, then added 63 μL of boron trifluoride diethyl etherate (0.5 mmole). The mixture was gently swirled on a shaker for 1 hr at room temperature. Methanol (2 mL) was added to the reaction mixture and the reaction was allowed to proceed another 5 min. The solution was drained and the resin was washed with THF, methanol, and dichloromethane. The resin was dried under vacuum before the measurement of the loading.

Measurement of the Loading Efficiency

Substitution levels, determined by the method of Meienhofer (Meienhofer, J.; Waki, M.; Heimer, E. P.; Lambros, T. J.; Makofske, R. C.; Chang, C. D. Int. J. Pept. Res. 1979, 13, 35-42.) through spectrophotometric detection of fulvene from resin aliquats, revealed a consistent range of values from 0.30-0.50 mmol/g, and were largely independent of the amino acid side chain (Table 5).

TABLE 5

| Fmoc AA | Substitution ^{#} | Yield * |
|---|---|---|
| Glycinol | 0.45 | 64.3 |
| Alaninol | 0.42 | 60.2 |
| D-alaninol | 0.52 | 74.6 |
| Argininol (Pbf) | 0.42 | 74.5 |
| Leucinol | 0.39 | 57.6 |
| Phenylalaninol | 0.40 | 60.4 |
| D-phenylalaninol | 0.49 | 74.0 |
| Prolinol | 0.42 | 61.3 |
| Tryptohanol | 0.36 | 55.8 |
| Lysinol (tBoc) | 0.35 | 55.7 |
| Threoninol (OtBu) | 0.48 | 73.0 |

^{#}in mmoles substrate per gram polystyrene
* based on percent of maximal theoretical substitution

The fulvene test was carried out as following:

Into a test tube containing certain amount of resin (usually 5 to 10 mg, 5.7 mg for the above prepared Fmoc-Thr(tBut)-ol-Wang resin as an example), added 4.0 mL 20% piperidine in DMF. Use 4.0 mL 20% piperidine in DMF in an empty test tube as blank. Over the 20 minutes, swirl the test tube with the resin two or three times to make sure all the resin has come in contact with the piperidine solution. Add DMF to both tubes to bring to a volume of 50 ml. Zero the spectropholometer at 301 nm with the blank. The absorbance of the solution is 0.427. Substitution calculation:

$$mmol \text{/} g = ( A301 \times Vol ( mL ) ) / ( 7.8 \times resin wt ( mg ) ) \\ = ( 0.427 \times 50 ) / ( 7.8 \times 5.7 ) - 0.48 mmole \text{/} g$$

### EXAMPLE 1

### Synthesis of Octreotide

The peptide synthesis can be carried out either in a manual fashion or by automated synthesis using the commercially available instruments. An ABI 433A synthesizer was used to assemble the primary sequence using a four-fold excess of DCC/HOBt (dicyclohexylcarbodiimide/hydroxybenztriazole) and each amino acid residue with a conventional protecting group scheme: Thr(OtBu), Cys(Trt), Lys(Boc), Trp(Boc). The above prepared Fmoc-threoninol(OtBu) Wang resin (a substitution level of 0.48 mmol/g, 0.2 mmole, 404 mg) was used to assemble the linear octreotide (dPhe-Cys-Phe-dTrp-Lys-Thr-Cys-Thr-ol). Following the assembly of the primary sequence the peptide was simultaneously deprotected and cleaved from the resin using a cleavage mixture of 45% TFA, 45% methylene chloride, 5% water and 5% thioanisole as scavengers. The yield of recovered peptide after purification was 37.8% based on the initial resin loading. Air oxidation of the free disulfide containing peptide in resulted in nearly quantitative disulfide bond formation and a 36.3% yield of octreotide after HPLC purification. HPLC conditons: Reverse-phased 300SB-C18 column, Zorbax-3.5 uM, 4.6×50 mm,. 2.0 mL/min, 0-65% of solvent B over 4 min. Solvent A: water with 0.1% TFA; Solvent B: acetonitrile with 0.1% TFA. The product was homogenous (single peak) on HPLC and the detected molecular weights by MS matched the calculated ones.

We claim:

1. A solid phase peptide synthetic method for the preparation of C-terminal alcohols, wherein the improvement is the use of trichloroacetimidate activated linker.

2. A process for preparing a C-terminal amino alcohol containing peptide comprising:
a. exposing the trichloroacetimidate modified Wang resin to an appropriate Fmoc protected amino alcohol to form a solid phase support;
b. reacting the support with an amino acid residue to form an attached peptide;
c. optionally repeating step b; and
d. cleaving the attached peptide to form said C-terminal amino alcohol containing peptide.

3. The process of claim 2 wherein the C-terminal amino alcohol containing peptide is octreotide.

4. The process of claim 2 or 3 wherein the Fmoc protected amino alcohol is Fmoc-Thr(tBut)-ol.

5. The process of claim 4 wherein step b is repeated sequentially with the following amino acid residues: Thr(OtBu), Cys(Trt), Thr(OtBu), Lys(Boc), Trp(Boc), Phe, Cys(Trt), and Phe.

6. A process for preparing a solid-phase support for the synthesis of a C-terminal amino alcohol containing peptide comprising
exposing trichloroacetimidate modified Wang resin to an appropriate Fmoc
protected amino alcohol to form said solid-phase support.

7. A process for preparing octreotide comprising:
a. exposing the trichloroacetimidate modified Wang resin to Fmoc-Thr(tBut)-ol to form a solid phase support;
b. reacting the support with Thr(OtBu) to form an attached peptide;
c. optionally repeating step b sequentially with the following amino acid residues: Cys(Trt), Thr(OtBu), Lys(Boc), Trp(Boc), Phe, Cys(Trt), and Phe; and
d. cleaving the attached peptide to form octreotide.
